(19) United States
(12) Patent Application Publication
(10) Pub. No.: US 2011/0054406 A1
(43) Pub. Date: Mar. 3, 2011

(54) VASCULATURE ENTRY CONFIRMATION MECHANISM

(75) Inventor: Austin Jason McKinnon, Herriman, UT (US)

(73) Assignee: BECTON, DICKINSON AND COMPANY, Franklin Lakes, NJ (US)

(21) Appl. No.: 12/552,592

(22) Filed: Sep. 2, 2009

(51) Int. Cl. A61B 17/34; H01R 3/08; H01S 4/00

(52) U.S. Cl. 604/168.01; 604/164.01; 439/179; 29/592.1

(57) ABSTRACT

A vascular entry confirmation mechanism that produces a signal when a cannula and/or catheter is properly placed in a blood vessel is described herein. Generally, the confirmation mechanism comprises a cannula (such as a venapuncture needle), a signaling element, a power source, electrical components to electrically connect the signaling element to the power source, and a switching mechanism, which is configured to close a circuit between the signaling element and power source when a fluid (e.g., blood), flows into the cannula. Accordingly, the confirmation mechanism is configured to produce a signal (e.g., an audible or a visual signal) when a patient's vasculature is punctured.

### BACKGROUND OF THE INVENTION

This disclosure relates generally to vascular access devices and associated methods. More specifically, this disclosure discusses an electrical vasculature entry confirmation mechanism. The confirmation mechanism can be used with a variety of vascular access devices, including intravenous (IV) catheter assemblies.

Generally, vascular access devices are used for communicating fluid with the vascular system of patients. For example, catheters are used for infusing fluid (e.g., saline solution, medicaments, and/or total parenteral nutrition) into a patient, withdrawing fluids (e.g., blood) from a patient, and/or monitoring various parameters of the patient's vascular system.

As mentioned, IV catheter assemblies are among the various types of vascular access devices. Over-the-needle peripheral IV catheters are a common IV catheter configuration. As its name implies, an over-the-needle catheter is mounted over an introducer needle having a sharp distal tip. The introducer needle is generally a venapuncture needle coupled to a needle assembly that helps guide the needle and facilitates its cooperation with the catheter. At least the inner surface of the distal portion of the catheter tightly engages the outer surface of the needle to prevent peelback of the catheter and, thereby, to facilitate insertion of the catheter into the blood vessel. The catheter and the introducer needle are often assembled so that the distal tip of the introducer needle extends beyond the distal tip of the catheter. Moreover, the catheter and needle are often assembled so that, during insertion, the bevel of the needle faces up, away from the patient's skin. The catheter and introducer needle are generally inserted at a shallow angle through the patient's skin into a blood vessel.

In some circumstances, the needle is configured to provide a “flashback” confirmation when the needle and/or catheter are properly placed in the blood vessel. Generally, this flashback entails the appearance of a small amount of blood, which exits the inner lumen of the needle through a notch feature that is defined in the needle's sidewalk As the blood exits the lumen, the blood becomes visible within the needle assembly or in an annular passage between the outer diameter of the needle and the inner diameter of the transparent catheter. Once an operator sees the flashback, the operator may determine that proper placement of the distal tip of the catheter into the blood vessel has occurred.

After determining flashback, the operator can withdraw the introducer needle from the catheter. In some instances, on order to remove the needle from the catheter, the operator applies pressure to the blood vessel by pressing down on the patient's skin over the blood vessel, distal to the introducer needle and the catheter. This finger pressure momentarily occludes the vessel, minimizing further blood flow through the introducer needle and the catheter. The operator may then withdraw the introducer needle from the catheter and leave the catheter in the patient's blood vessel.

In other instances, instead of using digital pressure to prevent blood spillage from the catheter during insertion and extraction from a blood vessel, some catheters are fitted with a small blood control seal or valve. Such a valve or seal is typically configured to close upon needle withdrawal, and to re-open upon connection so as to allow infusion.

While flashback confirmation provides a simple and fast method for determining proper needle and/or catheter placement, many current flashback confirmation mechanisms have challenges. For example, the notch feature in many, if not all, flashback-capable needles is configured to circumvent blood seals. Accordingly, as the needle is withdrawn from the catheter, some blood may be allowed to leak from the notch feature. In this manner, such needles may increase the risk of potentially dangerous blood exposure to operators, patients, and others.

In order to reduce this risk of unintentional blood exposure from flashback-capable needles, some have begun using such needles with a blood control seal that is longer than the distance between the needle's distal tip and the needle's notch feature. In such cases, when the needle is extracted from the catheter, the distal tip of the needle is drawn from the pressurized bloody side of the blood control seal and into the seal before the notch features exits the proximal side of the seal. Unfortunately, such seals tend to be relatively long, especially when used for larger gauge needles. Accordingly, the size of such seals may limit the number of devices with which the seals and needles can be used. In the alternative, devices, such as catheter adaptors, may have to be modified (e.g., lengthened) in order to be used with the elongated blood control seals and the notched, flashback-capable needles.

The present disclosure discusses a electrical mechanism for providing vasculature entry confirmation. Instead of requiring a notch feature to provide flashback confirmation, this vasculature entry confirmation mechanism comprises an electrical circuit that produced a signal when the needle and/or catheter assembly are properly placed in a blood vessel. Accordingly, described confirmation mechanism can reduce the likelihood of blood exposure that may be associated with notched flashback-capable needles.

### BRIEF SUMMARY OF THE INVENTION

The present application relates to a vascular entry confirmation mechanism that is designed to overcome some of the limitations known in the art. The confirmation mechanism may be used with any suitable device such as a cannula and/or a catheter. Additionally, while the confirmation mechanism may comprise any suitable component, the mechanism typically includes at least one signaling element, power source, electrical component for electrically connecting the signaling element to the power source, and switching mechanism that is configured to close a circuit between the power source and the signaling element when a fluid, such as blood, flows into the cannula. Accordingly, the confirmation mechanism is configured to produce a signal to indicate when a distal tip of the cannula and/or a catheter is properly inserted into a blood vessel.

Where the vascular entry device includes or is used with a cannula, the cannula may have any suitable component, including, a sharp or relatively sharp distal tip and an elongated tubular shaft comprising an inner lumen. Additionally, the cannula can comprise any suitable rigid tube having an inner lumen and a sharpened distal tip that is configured to puncture a patient's body and to draw off or introduce fluid into the patient. For example, the cannula may comprise a venapuncture needle, such as a phlebotomy needle, an arterial needle, a venous needle, and an introducer needle for use in an IV catheter assembly (e.g., an over-the-needle peripheral IV catheter assembly). Additionally, the cannula can have any characteristic that allows the cannula to fulfill its intended purpose and be used with the vasculature entry confirmation mechanism. For instance, the cannula may be any suitable length or gauge.

As mentioned above, the vasculature entry confirmation mechanism comprises at least one signaling element that is capable of producing a perceivable signal when the circuit between the signaling element and the power source is closed. Some examples of perceivable signals that can be produced by the signaling element may comprise an audio signal (e.g., one or more beeps, tones, words, or other sounds) or a visual signal (e.g., one or more light waves, images, or other visually perceptible signals). In some preferred implementations, the signaling element comprises a low-cost beeper or light, such as a light emitting diode (“LED”).

The power source may be selected from a variety of elements that are capable of providing the signaling element with a sufficient amount of electricity to produce a perceivable signal. Some examples of suitable power sources may include one or more batteries (e.g., a low-cost, commercially-available button-cell, hearing aid, or watch type battery), a capacitor, or an alternating current source (e.g., a municipal power grid). In some preferred implementations, however, the power source comprises a micro-battery or capacitor.

The electrical components in the vascular entry confirmation mechanism may comprise any components that are configured to electrically connect the signaling element to the power source. Some examples of suitable electrical components comprise electrically conductive wires, ribbons, strips, or other suitable electrically-conductive materials that extend between the power source and signaling element.

In some cases, the electrical components further comprise a first and a second electrical connector that are electrically insulated from each other when the inner lumen is dry (e.g., free from blood). While the first and the second electrical connectors can comprise any suitable components, in some cases, the first and the second electrical connectors each comprise conductive object (e.g., a wire or a ribbon) that is inserted into the cannula's inner lumen. In other cases, however, the first electrical connector comprises a conductive object that is inserted into the cannula's inner lumen and the cannula itself acts as the second electrical connector.

The switching mechanism may comprise any suitable components that are configured to close a circuit between the power source and the signaling element when a conductive fluid, such as blood, flows through the cannula's inner lumen. For instance, the switching mechanism may comprise a first non-insulated contact surface on the first electrical connector and an adjacent, second, non-insulated contact surface on the second electrical connector. In such instances, as blood flows into the inner lumen, the blood may bridge a gap between the first and the second contact surface and, thereby, close the circuit between the signaling element and the power source.

In some cases, in order to reduce the chance of having interstitial fluid falsely activate the signaling element, the first and/or the second contact surfaces are disposed proximal to the proximal-most part of the cannula's bevel. Accordingly, the circuit may only be closed when significant amounts of blood flow into the inner lumen.

The described vascular access device may be used with any suitable device or assembly. For example, the device may be used with a phlebotomy needle assembly, an arterial needle assembly, a venous needle assembly, or an IV catheter assembly (hereinafter, collectively referred to as “needle assembly”). In this example, the various components of the vascular access device may be disposed in any suitable location. Indeed, in some instances, the cannula and all of the components of the vascular entry confirmation mechanism are disposed on or in the needle assembly. In such instances, the entire vascular entry confirmation mechanism can be disposed of with the cannula, after a single use.

In other instances, only a portion of the vascular entry confirmation device is disposed on or in the needle assembly. In one example, while the first and the second electrical connectors are at least partially exposed within the cannula in the needle assembly, the power source and signaling element are disposed in a separate device that is capable of being electrically coupled to and uncoupled from the first and second electrical connector. In this example, the cannula and the first and second electrical connectors are configured to be disposed of after a single use and the separate device comprising the power source and the signaling element is configured to be used multiple times.

### BRIEF DESCRIPTION OF THE SEVERAL VIEWS OF THE DRAWINGS

In order that the manner in which the above-recited and other features and advantages of the invention are obtained and will be readily understood, a more particular description of the invention briefly described above will be rendered by reference to specific embodiments thereof, which are illustrated in the appended drawings. Understanding that these drawings depict only typical embodiments of the invention and are not, therefore, to be considered to be limiting of its scope, the invention will be described and explained with additional specificity and detail through the use of the accompanying drawings in which:

FIG. 1 illustrates a side cross section view of a representative embodiment of a vasculature entry confirmation mechanism;

FIG. 2 illustrates a diagram of a representative embodiment of an electrical circuit for the vasculature entry confirmation mechanism of FIG. 1;

FIG. 3 illustrates a side cross section view of a representative embodiment of the vasculature entry confirmation mechanism;

FIG. 4 illustrates a perspective view of a representative embodiment of a needle comprising a first and a second electrical connector;

FIG. 5 illustrates a cross section view of a representative embodiment of the vasculature entry confirmation mechanism;

FIG. 6 illustrates a side cross section view of a representative embodiment of a catheter assembly comprising the vasculature entry confirmation mechanism;

FIG. 7 illustrates a side cross section view of a representative embodiment of a catheter assembly comprising some components of the vasculature entry confirmation mechanism and a block diagram of a separate device comprising additional components of the confirmation mechanism; and

FIG. 8 illustrates a graph of representative experimental results that illustrates a level of signal potential that is produced when simulated blood fills the tip of a cannula from the vasculature entry confirmation mechanism.

### DETAILED DESCRIPTION OF THE INVENTION

The presently preferred embodiments of the described invention will be best understood by reference to the Figures, wherein like parts are designated by like numerals throughout. It will be readily understood that the components of the present invention, as generally described and illustrated in the accompanying Figures, could be arranged and designed in a wide variety of different configurations. Thus, the following more detailed description of the embodiments of the vasculature entry confirmation mechanism, as represented in the Figures, is not intended to limit the scope of the invention, as claimed, but is merely representative of some presently preferred embodiments.

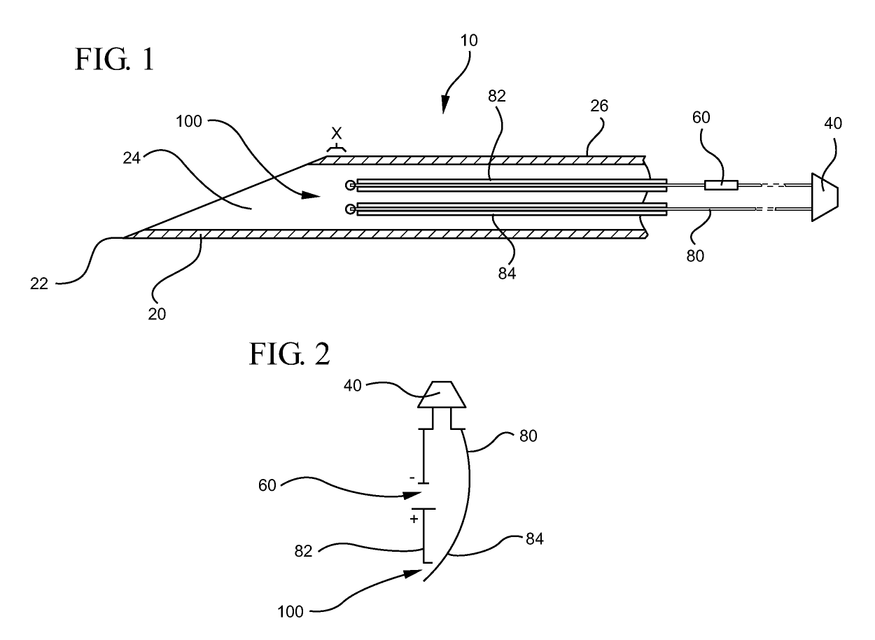

Generally, this application relates to a vasculature entry confirmation mechanism that produces a signal when a cannula and/or catheter is properly placed in a fluid containing compartment (e.g., a blood vessel) within a patient. While the described confirmation mechanism may comprise any suitable component, FIG. 1 shows a representative embodiment in which the vasculature entry confirmation mechanism 10 comprises a cannula (e.g., needle 20), a signaling element 40, a power source 60, electrical components 80 (e.g., first 82 and second 84 electrical connector) for electrically connecting the signaling element 40 to the power source 60, and a switching mechanism 100 that is configured to close a circuit between the power source and the signaling element with a fluid, such as blood, flows into the cannula. A representative embodiment of a circuit diagram corresponding to the confirmation mechanism of FIG. 1 is provided in FIG. 2. To provide a better understanding of the vasculature entry confirmation device, each of its aforementioned components is described below in more detail.

Where the confirmation mechanism comprises a cannula, the cannula may comprise virtually any rigid tube that includes a sharpened distal tip and which is configured to puncture a patient's body, to access an intended space, and to withdraw or to introduce a material (e.g., a fluid) from or into the intended space. One example of a suitable cannula comprises a venapuncture needle, In this example, the venapuncture needle may include virtually any suitable venapuncture needle, including an introducer needle for use in an IV catheter assembly (e.g., an over-the-needle peripheral IV catheter assembly), a venous needle, an arterial needle, and the like. For simplicity, however, the cannula of the vasculature entry confirmation mechanism is described below with reference to an introducer needle.

The introducer needle may have any component that is suitable for use with an IV catheter assembly. For instance, FIG. 1 shows a representative embodiment in which the introducer needle 20 comprises a sharpened distal tip 22, an inner lumen 24, and an elongated tubular shaft 26. Moreover, each component of the needle may have any suitable characteristic. For example, the distal tip of the needle may comprise a standard bevel, a short bevel, a true short bevel, a bias grind point, a vet point, a lancet point, a deflected point (anti-coring), or another suitable known or novel needle point. Additionally, the lumen and the elongated tubular shaft may be any suitable size. For example, the needle may be any suitable length or any suitable gauge (e.g., from about a 7 to about a 33 on the Stubs scale) that allows it to be used as the introducer needle in an IV assembly.

As mentioned above, the confirmation mechanism comprises at least one signaling element. The signaling element may comprise virtually any device that allows the confirmation mechanism to fulfill its intended purpose and which is capable of producing a human-perceivable signal when the circuit between the signaling element and the power source is closed.

In some embodiments, the signaling element comprises one or more devices that produce an audible signal, a visual signal, or both. Some examples of suitable devices that produce an audible signal include a speaker, a buzzer, a beeper, or another device that produces an audible sound (e.g., one or more beeps, tones, noises, words, sounds, etc.). In some preferred embodiments, the device comprises a low-cost beeper that is sized to fit within a catheter assembly.

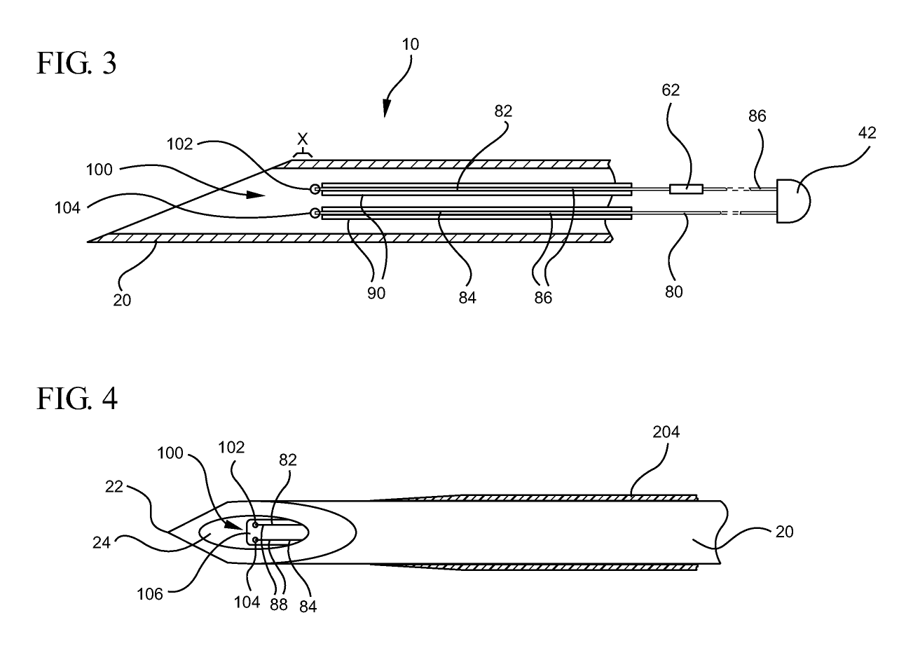

Some examples of suitable devices that produce a visual signal include a light (e.g., an LED, an incandescent light, etc.), a visual display (e.g., a monitor, a liquid crystal display (“LCD”), or another electrical component that is capable of providing visibly perceivable light waves, images, or other visual indications that the circuit is closed. By way of non-limiting illustration, FIG. 3 shows a representative embodiment in which the signaling element comprises a low-cost LED 42 that configured to be disposed in the catheter assembly (not shown in FIG. 3).

The confirmation mechanism further includes a power source. The power source may comprise any suitable device that is capable of powering the signaling element and allowing the confirmation mechanism to fulfill its intended purpose. For instance, the power source may comprise a direct current (“DC”) power source. Some examples of suitable DC power sources include a battery (e.g., a low-cost, commercially-available button-cell, hearing-aid, or watch type battery) or a capacitor. Indeed, in some presently preferred embodiments, the power source is a DC battery or capacitor that is contained within the catheter assembly. By way of illustration, FIG. 3 shows one embodiment in which the power source comprises a low-cost micro-battery 62. Nevertheless, in some other embodiments, the power source comprises an alternating current (“AC”) power source. An example of a suitable AC power source is a suitable connection (e.g., a plug, transformer, and/or another suitable component) to a power grid, such as a municipal power supply.

FIG. 3 also shows that the confirmation mechanism 10 comprises one or more electrical components 80 that are configured to electrically connect the signaling element (e.g., LED 42) to the power source (e.g., micro-battery 62). Indeed, the confirmation mechanism may comprise any electrically-conductive component that is suitable to electrically connect the signaling element to the power source when a conductive fluid, such as blood, flows into the cannula's inner lumen. For instance, an electrically-conductive wire, ribbon, strip, trace on a printed circuit wire board (“PCB”), or another conductive component may extend between the power source and the signaling element. By way of non-limiting illustration, FIG. 3 shows an embodiment in which a wire 86 extends between the battery 62 and the LED 42.

In some embodiments, the electrical components further comprise a first electrical connector and a second electrical connector, which are electrically insulated from each other when the cannula's lumen is free from a conductive fluid (e.g., blood). In some embodiments, one of the electrical connectors (e.g., either the first or the second electrical connector) is electrically connected to the power source and the other electrical connector is electrically connected to the signaling element.

The first and second electrical connectors may comprise practically any electrically-conductive component that allows the confirmation mechanism to function as intended. In one example, the first and the second electrical connectors each comprise an electrically conductive object (e.g., a small wire, ribbon, etc.) that is inserted into the cannula's lumen without unduly occluding the lumen. For instance, FIG. 3 shows an embodiment in which the first 82 and the second 84 electrical connectors each comprise a small gauge magnet wire 86 that is inserted into the cannula's lumen 24. Similarly, FIG. 4 shows a representative embodiment in which the first 82 and the second 84 electrical connectors comprise micro-printed traces 88.

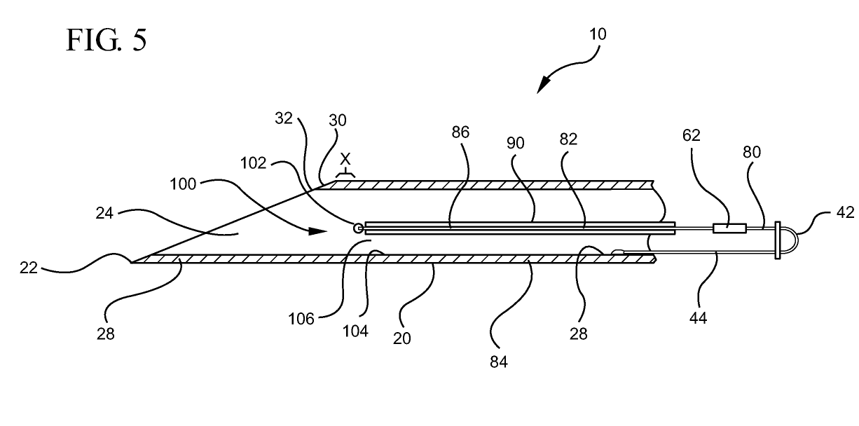

In contrast, FIG. 5 shows a representative embodiment in which the first electrical connector 82 comprises a magnet wire 86 and the second electrical connector 84 comprises the cannula 20 itself. In such an embodiment, the cannula may be electrically connected to the various components of the confirmation mechanism in any suitable manner. For instance, the cannula can be soldered, joined with a conductive epoxy, mechanically clamped to, or otherwise be electrically connected to the power source, the signaling element, and/or a conductive element that extends to the power source or the signaling element. By way of non-limiting illustration, FIG. 5 shows an embodiment in which a probe 44 from the LED 42 is soldered to the sidewall 28 of the cannula 20.

When the inner lumen is free from a conductive fluid, the first and the second electrical connectors may be electrically isolated from each other by any suitable material. For example, one or more of the electrical connectors may be at least partially shielded from the other electrical conductor by an insulative material (e.g., a polyethylene, a plastic, a glass, TEFLON®, or another dielectric) and/or by physical separation. By way of illustration, FIG. 5 illustrates an embodiment in which the first 82 and the second 84 electrical connectors are physically and electrically separated in the dry inner lumen 24 through the use of a dielectric sheath 90.

The confirmation mechanism also comprises a switching mechanism that is configured to close the circuit to cause the signaling element to produce a signal when blood, or another conductive fluid, flows into the cannula's lumen. The switching mechanism may comprise any component that allows the confirmation mechanism to be used as intended. Some examples of suitable switching mechanisms may include a capacitance touch switch, a resistance touch switch, and a switch comprising a first non-insulated contact surface on the first electrical connector and a second non-insulted contact surface on the second electrical connector.

In some currently preferred embodiments, FIG. 5 shows that the switching mechanism 100 comprises a first 102 and a second 104 non-insulated contact surface (e.g., the inner surface of the cannula's sidewall 28) on the first 82 and second 84 electrical connectors, respectively. In such embodiments, when blood (not shown) flows into the inner lumen 24, the blood may bridge the gap 106 between the first 102 and the second 104 contact surfaces and, thereby, close the circuit.

Where the switching mechanism comprises the first and the second non-insulated contact surfaces of the first and second electrical connectors, the non-insulated surfaces may be disposed any suitable distance from the cannula's distal tip. In some embodiments, however, to reduce the likelihood of having interstitial fluid falsely activate the signaling element, at least one of the non-insulated contact surfaces is disposed proximal to the proximal-most part of the cannula's bevel. Accordingly, in such embodiments, the circuit may only be closed when a sufficient amount of blood flows into the lumen to reach the contact surfaces and bridge the gap between them.

While the first and/or second contact surface may be located any suitable distance proximal to the proximal-most end of the cannula's bevel, in some embodiments, the first and/or the second contact surface is proximally disposed less than about 1 centimeter from the proximal-most part of the bevel. In another embodiment, at least one contact surface is proximally disposed less than about 5 millimeters from the bevel's proximal-most end. In still another embodiment, however, at least one contact surface is proximally disposed within less than about 3 millimeters from the bevel's proximal-most end. For example, FIG. 5 illustrates an embodiment in which the contact surface 102 of the first electrical connector 82 is disposed a distance x of about 2 millimeters, proximal to the proximal-most part 30 of the cannula's bevel 32.

In addition to the previously mentioned embodiments of the vasculature entry confirmation mechanism, the mechanism can be varied in any suitable manner. In one example, the cannula may comprise any suitable component. For instance, the cannula can comprise a cannula feature, such as a crimp feature, a ferrule feature, a one-way barb, or another component that is configured to be captured by a known or novel cannula feature capture mechanism to limit the cannula's movement in at least the proximal direction with respect to the capture mechanism.

In another example, in addition to the signaling element, the power source, and the electrical components, the vasculature entry confirmation mechanism may comprise any additional electrical element that allows the confirmation mechanism to fulfill its intended purpose. By way of example, the confirmation mechanism may comprise one or more resistors, transistors, transformers, integrated circuits, etc.

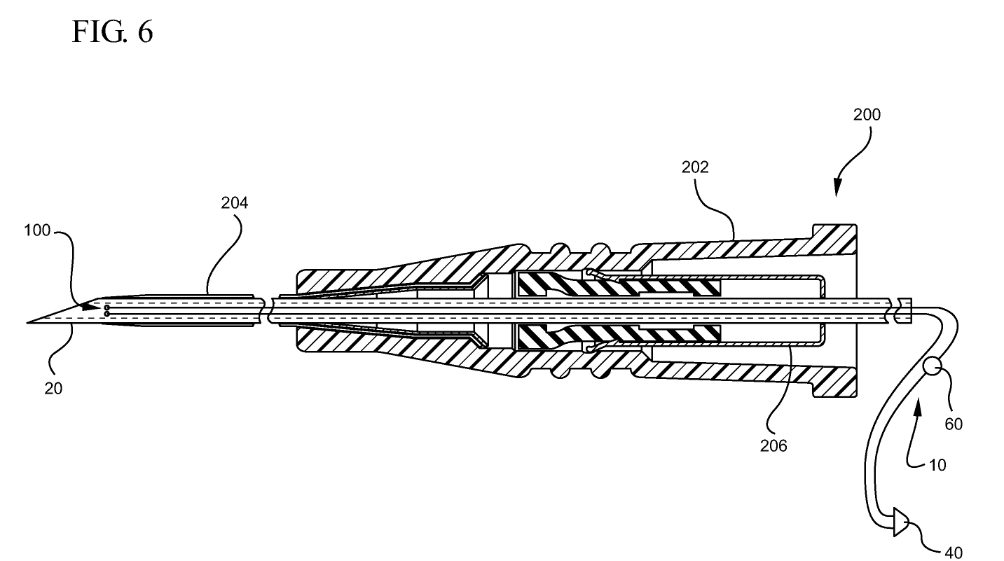

Moreover, the confirmation mechanism may be used with any suitable device or system. Indeed, as previously stated, the confirmation mechanism may be used with any suitable catheter assembly. By way of illustration, FIG. 6 shows that in a representative embodiment, the confirmation mechanism 10 may be used with a catheter assembly 200 comprising a catheter adapter 202, a catheter 204, and a cannula capture mechanism 206. While the entire catheter assembly is not illustrated, the skilled artisan will recognize that in this embodiment, the power source 60 and the signaling element 40 can be disposed in or on the catheter assembly 200 in any suitable manner.

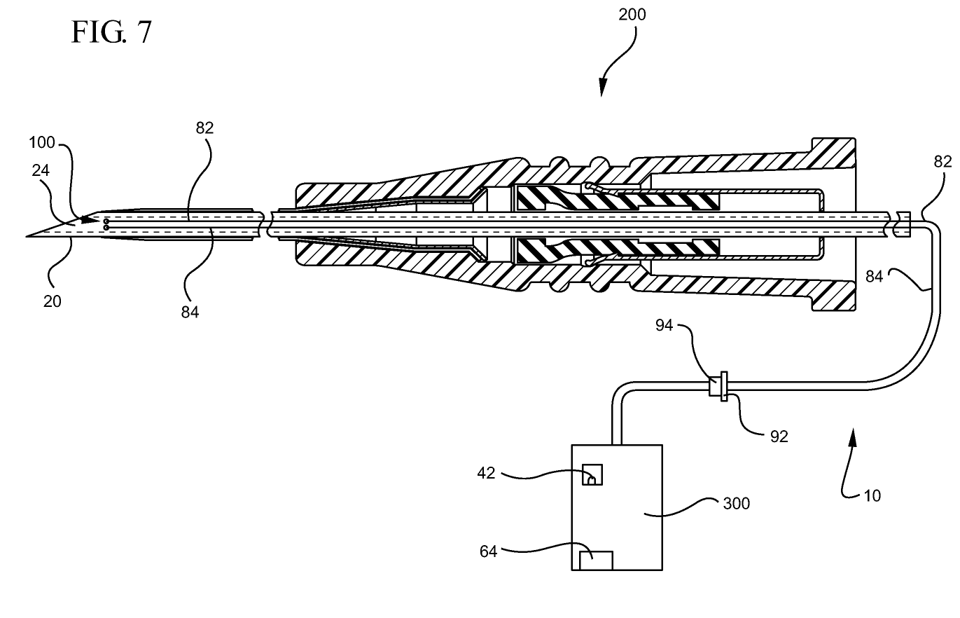

In other embodiments, however, only some of the confirmation mechanism's components are disposed in the catheter assembly and the rest of its components are disposed separate to the catheter assembly. In such embodiments, the components may be disposed in any suitable manner that allows the confirmation mechanism to operate as intended. By way of non-limiting illustration, FIG. 7 shows a representative embodiment in which the first 82 and the second 84 electrical connectors are at least partially disposed within the catheter assembly 200.

Specifically, FIG. 7 shows that the first 82 and the second 84 electrical connectors extend proximally through the cannula's inner lumen 24 and terminate at a first electrical connection point 92 (e.g., a plug, a blade connector, etc.). Additionally, FIG. 7 shows that a second device 300 comprises the signaling element (e.g., LED 42), the power source (e.g., alkaline batteries 64, and a second electrical connection point 94 that is configured to mate with the first electrical connection point 92. In the embodiment shown in FIG. 7, the needle 20, the first 82 and second 84 connectors, as well as the first electrical connection point 92 may be discarded after a single use. In contrast, the second device 300 may be reused many times with many different needles. Additionally, because the second device is not discarded after a single use, the second device may comprise more expensive components than would be practical in embodiments in which the entire confirmation mechanism is configured to be disposed after a single use.

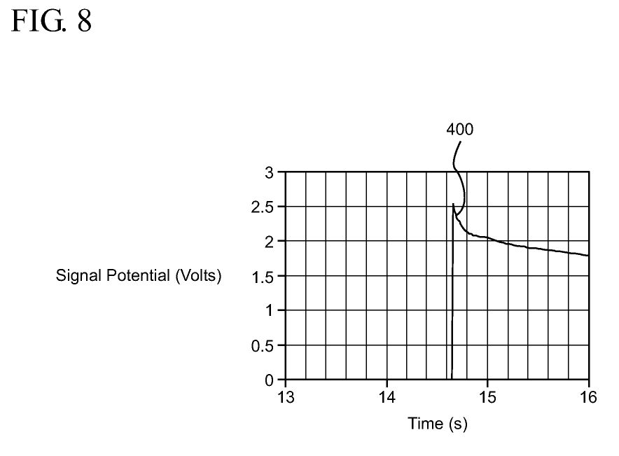

The described vasculature entry confirmation mechanism may be used in any suitable manner. For example, an operator may use the distal point of the needle to puncture the skin of a patient and force an over-the-needle peripheral IV catheter into a blood vessel. Once the needle has penetrated the blood vessel and blood flows into the cannula's lumen, the circuit is closed and, as shown in FIG. 8, signal potential 400 increases and the signal is produced. At that point, the cannula may be extracted from the catheter, and the cannula and entire confirmation mechanism (depending on the embodiment) may be discarded.

The vasculature entry confirmation mechanism may offer several benefits and advantages over certain conventional, flashback-capable needles. In one example, because the described confirmation mechanism does not necessarily require a notch through which blood can exit the inner lumen to provide flashback confirmation, the described confirmation mechanism may provide a mechanism to determine proper needle and/or catheter placement without the same risks of blood exposure that are associate with notched needles. In another example, because the described confirmation mechanism does not necessarily require a notch in the cannula, suitable blood control seals can be shorter than comparable seals in assemblies in which the needle comprises a notch. As a result, the described confirmation mechanism may be used in a larger variety of catheter assemblies.

The present invention may be embodied in other specific forms without departing from its structures, methods, or other essential characteristics as broadly described herein and claimed hereinafter. The described embodiments and examples are all to be considered in every respect as illustrative only, and not as being restrictive. The scope of the invention is, therefore, indicated by the appended claims, rather than by the foregoing description. All changes that come within the meaning and range of equivalency of the claims are to be embraced within their scope.

1. A vasculature entry confirmation mechanism, comprising:
a cannula with an inner lumen;
a power source;
a signaling element;
a first electrical connector; and
a second electrical connector,
wherein the first electrical connector is disposed within the inner lumen, and
wherein a circuit between the power source and the signaling element remains open until an electrically conductive fluid flows into the inner lumen and electrically connects the first electrical connector to the second electrical connector to close the circuit and produce a signal.

2. The mechanism of claim 1, wherein the signaling element produces a visible signal when the circuit closes.

3. The mechanism of claim 1, wherein the signaling element produces an audible signal when the circuit closes.

4. The mechanism of claim 1, wherein the second electrical connector comprises the cannula.

5. The mechanism of claim 1, wherein the first electrical connector and the second electrical connector are disposed within the inner lumen.

6. The mechanism of claim 1, wherein the power source, the signaling element, and the electrical connectors are disposed on or in a catheter assembly.

7. The mechanism of claim 1, wherein the power source and the signaling element are disposed on a device that is separate from a catheter assembly comprising the first electrical connector and the second electrical connector.

8. The mechanism of claim 2, wherein the signaling element comprises a light emitting diode.

9. The mechanism of claim 1, wherein the first electrical connector is disposed proximal to the proximal-most end of a bevel of the cannula.

10. A catheter assembly, comprising:
a catheter adapter; and
a vasculature entry confirmation mechanism comprising:
a cannula with an inner lumen;
a power source; and
a signaling element,
wherein a circuit between the power source and the signaling element remains open until an electrically conductive fluid flows into the inner lumen and closes the circuit to produce a signal.

11. The assembly of claim 10, wherein the signaling element produces a visible signal when the circuit closes.

12. The assembly of claim 10, wherein the signaling element produces an audible signal when the circuit closes.

13. The assembly of claim 10, wherein the vasculature entry confirmation mechanism further comprises a first electrical connector and a second electrical connector, and wherein the first electrical connector is disposed within the inner lumen.

14. The assembly of claim 13, wherein the second electrical connector is disposed within the inner lumen.

15. The assembly of claim 13, wherein the second electrical connector comprises the cannula.

16. A method for making a vasculature entry confirmation mechanism, the method comprising:
providing a cannula with an inner lumen;
providing a power source;
providing a signaling element; and
providing electrical components that connect the power source to the signaling element,
wherein a circuit between the power source and the signaling element is open until an electrically conductive fluid flows into the inner lumen and closes the circuit to produce a signal.

17. The method of claim 16, wherein the electrical components comprise a first electrical connector that is disposed within cannula, proximal to a proximal-most end of a bevel of the cannula.

18. The method of claim 17, wherein the electrical components further comprise a second electrical connector that comprises the cannula.

19. The method of claim 16, wherein the signaling element produces a visible signal when the circuit closes.

20. The method of claim 16, wherein the electrical components comprise a first electrical connector and a second electrical connector, and wherein the first electrical connector and the second electrical connector are disposed within the inner lumen of the cannula.
